SEQUENCE LISTING (6 sequences)

SEQ ID NO: 1 - protein, 562 aa, Acidothermus cellulolyticus
MPRALRRVPGSRVMLRVGVVVAVLALVAALANLAVPRPARAAGGGYWHTSGREILDANNVPVRIAGINWFGFETCNYVVHGLWSRDYRSMLDQIKSLGYNTIRLPYSDDILKPGTMPNSINFYQMNQDLQGLTSLQVMDKIVAYAGQIGLRIILDRHRPDCSGQSALWYTSSVSEATWISDLQALAQRYKGNPTVVGFDLHNEPHDPACWGCGDPSIDWRLAAERAGNAVLSVNPNLLIFVEGVQSYNGDSYWWGGNLQGAGQYPVVLNVPNRLVYSAHDYATSVYPQTWFSDPTFPNNMPGIWNKNWGYLFNQNIAPVWLGEFGTTLQSTTDQTWLKTLVQYLRPTAQYGADSFQWTFWSWNPDSGDTGGILKDDWQTVDTVKDGYLAPIKSSIFDPVGASASPSSQPSPSVSPSPSPSPSASRTPTPTPTPTASPTPTLTPTATPTPTASPTPSPTAASGARCTASYQVNSDWGNGFTVTVAVTNSGSVATKTWTVSWTFGGNQTITNSWNAAVTQNGQSVTARNMSYNNVIQPGQNTTFGFQASYTGSNAAPTVACAAS

SEQ ID NO: 2 - DNA, 1689 bp, Acidothermus cellulolyticus
  source 1..1689: mol_type = other DNA
gtgccgcgcgcattgcggcgagtgcctggctcgcgggtgatgctgcgggtcggcgtcgtcgtcgcggtgctggcattggttgccgcactcgccaacctagccgtgccgcggccggctcgcgccgcgggcggcggctattggcacacgagcggccgggagatcctggacgcgaacaacgtgccggtacggatcgccggcatcaactggtttgggttcgaaacctgcaattacgtcgtgcacggtctctggtcacgcgactaccgcagcatgctcgaccagataaagtcgctcggctacaacacaatccggctgccgtactctgacgacattctcaagccgggcaccatgccgaacagcatcaatttttaccagatgaatcaggacctgcagggtctgacgtccttgcaggtcatggacaaaatcgtcgcgtacgccggtcagatcggcctgcgcatcattcttgaccgccaccgaccggattgcagcgggcagtcggcgctgtggtacacgagcagcgtctcggaggctacgtggatttccgacctgcaagcgctggcgcagcgctacaagggaaacccgacggtcgtcggctttgacttgcacaacgagccgcatgacccggcctgctggggctgcggcgatccgagcatcgactggcgattggccgccgagcgggccggaaacgccgtgctctcggtgaatccgaacctgctcattttcgtcgaaggtgtgcagagctacaacggagactcctactggtggggcggcaacctgcaaggagccggccagtacccggtcgtgctgaacgtgccgaaccgcctggtgtactcggcgcacgactacgcgacgagcgtctacccgcagacgtggttcagcgatccgaccttccccaacaacatgcccggcatctggaacaagaactggggatacctcttcaatcagaacattgcaccggtatggctgggcgaattcggtacgacactgcaatccacgaccgaccagacgtggctgaagacgctcgtccagtacctacggccgaccgcgcaatacggtgcggacagcttccagtggaccttctggtcctggaaccccgattccggcgacacaggaggaattctcaaggatgactggcagacggtcgacacagtaaaagacggctatctcgcgccgatcaagtcgtcgattttcgatcctgtcggcgcgtctgcatcgcctagcagtcaaccgtccccgtcggtgtcgccgtctccgtcgccgagcccgtcggcgagtcggacgccgacgcctactccgacgccgacagccagcccgacgccaacgctgacccctactgctacgcccacgcccacggcaagcccgacgccgtcaccgacggcagcctccggagcccgctgcaccgcgagttaccaggtcaacagcgattggggcaatggcttcacggtaacggtggccgtgacaaattccggatccgtcgcgaccaagacatggacggtcagttggacattcggcggaaatcagacgattaccaattcgtggaatgcagcggtcacgcagaacggtcagtcggtaacggctcggaatatgagttataacaacgtgattcagcctggtcagaacaccacgttcggattccaggcgagctataccggaagcaacgcggcaccgacagtcgcctgcgcagcaagttaa

SEQ ID NO: 3 - protein, 513 aa, Trichoderma koningii
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNRGTTTTRRPATTTGSSPGPTQSHYGQCGGIGYSGPTVCASGTTCQVLNPYYSQCL

SEQ ID NO: 4 - DNA, 1542 bp, Trichoderma koningii
  source 1..1542: mol_type = other DNA
atgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaaccgtggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaa

SEQ ID NO: 5 - protein, 471 aa, Trichoderma reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 6 - DNA, 1416 bp, Trichoderma reesei
  source 1..1416: mol_type = other DNA
atgattgtcggcattctcaccacgctggctacgctggccacactcgcagctagtgtgcctctagaggagcggcaagcttgctcaagcgtctggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggatcgggaaccgctacgtattcaggcaacccttttgttggggtcactccttgggccaatgcatattacgcctctgaagttagcagcctcgctattcctagcttgactggagccatggccactgctgcagcagctgtcgcaaaggttccctcttttatgtggctagatactcttgacaagacccctctcatggagcaaaccttggccgacatccgcaccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatatccggaccctcctggttattgagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagctcttcgcggattggcaaccaatgtcgccaactacaacgggtggaacattaccagccccccatcgtacacgcaaggcaacgctgtctacaacgagaagctgtacatccacgctattggacctcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactcccactgtgcgctcccagatgccttgcaaccggcgcctcaagctggtgcttggttccaagcctactttgtgcagcttctcacaaacgcaaacccatcgttcctgtaa

(19) United States
(12) Patent Application Publication
(10) Pub. No.: US 2024/0392333 A1
(43) Pub. Date: Nov. 28, 2024

(54) METHODS AND COMPOSITIONS FOR INCREASING GLUCOSE YIELD FROM GRAIN

(71) Applicant: GREENLAB, INC., Jonesboro, AR (US)

(72) Inventors: Elizabeth E. Hood, Jonesboro, AR (US); John A. Howard, San Luis Obispo, CA (US)

(21) Appl. No.: 18/672,521

(22) Filed: May 23, 2024

Related U.S. Application Data
(60) Provisional application No. 63/504,235, filed on May 25, 2023.

(51) Int. Cl. C12P 19/02; C12P 7/06; C12P 19/14

(52) U.S. Cl. CPC C12P 19/02; C12P 7/06; C12P 19/14

(57) ABSTRACT

The present disclosure provides methods of converting cellulose in grain to glucose for ethanol production. By providing cost-effective cellulases expressed in the grain, the methods increase glucose yield compared to the use of starch degrading amylase enzymes alone.

### CROSS REFERENCE TO RELATED APPLICATIONS

This application claims priority to provisional application U.S. Ser. No. 63/504,235, filed May 25, 2023, which is incorporated herein by reference in its entirety.

### SEQUENCE LISTING XML

The instant application contains a sequence listing, which has been submitted in XML file format by electronic submission and is hereby incorporated by reference in its entirety. The XML file, created on May 22, 2024, is named P14418US01.xml and is 12,211 bytes in size.

### TECHNICAL FIELD

The present disclosure relates to methods of saccharification of polysaccharides in grain, for increased production of glucose from grain for ethanol production.

### BACKGROUND

Energy is important to modern human existence from transportation, electricity, heat, and cooling. Most energy produced in the world today is from fossil fuels—oil, coal, and natural gas. However, because of the dramatic changes in climate due to the belching of CO₂ from fossil fuels, alternatives have become highly important. First generation biofuels for transportation comprised ethanol from starch and biodiesel from oils. Second and third generation fuels comprise ethanol from cellulose and renewable diesel among others. Ethanol production in the US is most commonly from corn using a dry- or wet-mill process. Because of lower capital costs, most are dry milled, which is whole corn ground into flour. Co-products of this process after fermentation are distillers' grains and carbon dioxide.

Thirty to 40% of corn in the United States is used for ethanol production, depending on the year. To increase ethanol production without increasing acreage or percentage of crop to sugar production for fermentation, increased glucose output from current facilities is warranted. Current methods use only starch degrading enzymes, α-amylase and amyloglucosidase, to produce glucose. Starch is 60-62% of the kernel weight. Cellulose in the kernel offers another 13-15% of the kernel weight as glucose. Thus, if the cellulose could also be degraded, the yield of glucose and thus the yield of ethanol could be increased by several percent.

### SUMMARY

Methods of producing glucose are provided. In certain embodiments, the methods comprise contacting milled grain with one or more cellulases under conditions sufficient to hydrolyze cellulose in the milled grain to glucose. In certain embodiments, the methods further comprise contacting the milled grain with one or more amylases to hydrolyze starch in the milled grain to glucose. In certain embodiments, the milled grain comprises a heterologous cellulase. For example, the milled grain can be obtained from a transgenic plant comprising a heterologous polynucleotide encoding the cellulase. In certain embodiments, the heterologous cellulase is an endoglucanase and/or a cellobiohydrolase. In certain embodiments, the milled grain is maize grain. The methods of the disclosure provide increased glucose yield relative to a method without the one or more cellulases.

Methods of producing ethanol are also provided. The methods comprise contacting milled grain with one or more cellulases and one or more amylases under conditions sufficient to hydrolyze cellulose and starch in the milled grain to glucose; and incubating the glucose with at least one fermenting microorganism under conditions in which ethanol is produced.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE DRAWINGS

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1 shows cellulose digestion using cellulase from Trichoderma reesei (CELLUCLAST®) plus and minus β-glucosidase.

FIG. 2 shows degradation of cellulose and starch from control flour. The cellulase treatment was 15 hours prior to heat treatment and adding amylases. Samples were incubated at 50° C. and rotated end over end. The control sample contained cellulase and β-glucosidase only.

FIG. 3 shows glucose released from corn flour with a single transgenic enzyme incubated with β-glucosidase for 7 days. Control is non-transgenic flour incubated with β-glucosidase. E1=endoglucanase; C1=cellobiohydrolase I; C2=cellobiohydrolase II.

FIG. 4 shows glucose released with a combination of two flours at 50 mg each and a combination of three flours at 35 mg each (E1+C1+C2). One mL of sodium acetate buffer pH=5+5 μl of β-glucosidase. E1=endoglucanase; C1=cellobiohydrolase I; C2=cellobiohydrolase II.

FIG. 5 shows glucose released from dilutions of E1 flour with control flour. Control is control flour with β-glucosidase only.

FIG. 6 shows glucose released from 50:50 E1 flour to control flour plus β-glucosidase prior to heating starch and adding amylases.

FIG. 7 shows glucose released from E1 flour heated to 85° C. for 10 minutes prior to incubation with amylases for 24 hours.

FIG. 8 shows glucose released using different sources of β-glucosidase. The treatments for each ID are summarized in Table 1.

FIG. 9 shows glucose released with different ratios of units of E1:CBH I:CBH II for cellulose degradation. The treatments for each ID are summarized in Table 3.

FIG. 10 shows glucose released when enzymes were heated prior to digestion holding time.

FIG. 11 shows glucose released from the digestion of cellulose in the mash that remains after saccharification and fermentation of corn grain.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Thus, for example, reference to “an enzyme” includes a single enzyme, as well as two or more enzymes; reference to “a plant” includes one plant, as well as two or more plants; and so forth. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

“Amylase” is meant to include any amylase, such as glucoamylases, α-amylases, and β-amylases. “Amylase” shall mean an enzyme that is, among other things, capable of catalyzing the degradation of starch. Amylases are hydrolases that cleave the α-D-(1→4) O-glycosidic linkages in starch. Generally, α-amylases (EC 3.2.1.1; α-D-(1→4)-glucan glucanohydrolase) are defined as endo-acting enzymes cleaving α-D-(1→4) β-glycosidic linkages within the starch molecule in a random fashion. In contrast, the exo-acting amylolytic enzymes, such as β-amylases (EC 3.2.1.2; α-D-(1→4)-glucan maltohydrolase) and some product-specific amylases like maltogenic α-amylase (EC 3.2.1.133) cleave the starch molecule from the non-reducing end of the substrate. β-Amylases, α-glucosidases (EC 3.2.1.20; α-D-glucoside glucohydrolase), glucoamylases (EC 3.2.1.3; α-D-(1-+4)-glucan glucohydrolase), and product-specific amylases can produce malto-oligosaccharides of a specific length from starch.

The term “β-glucosidase” is defined herein as a β-D-glucoside glucohydrolase (E.C. 3.2.1.21) which catalyzes the hydrolysis of cellobiose and shorter cello-oligosaccharides with the release of β-D-glucose.

The term “cellulase” refers to a category of enzymes capable of hydrolyzing cellulose β-1,4-glucan or β-D-glucosidic linkages) polymers to shorter cello-oligosaccharide oligomers, cellobiose and/or glucose.

The term “cellobiohydrolase” (CBH) refers to a group of cellulases classified as EC 3.2.1.91. These enzymes are also known as exoglucanases or exo-cellobiohydrolases. CBH enzymes hydrolyze cellobiose from the reducing or non-reducing end of cellulose. In general a CBHI type enzyme preferentially hydrolyzes cellobiose from the reducing end of cellulose and a CBHII type enzyme preferentially hydrolyzes the non-reducing end of cellulose.

“Cellulolytic activity” encompasses exoglucanase activity, endoglucanase activity or both types of enzyme activity, as well as β-glucosidase activity.

The term “endoglucanase” refers to a group of cellulases classified as EC 3.2.1.4. An endoglucanase enzyme hydrolyzes internal β-1,4 glucosidic bonds of the cellulose.

The term “contacting” refers to the placing of the respective enzyme(s) in sufficiently close proximity to the respective substrate to enable the enzyme(s) to convert the substrate to the end-product. Those skilled in the art will recognize that mixing solutions of the enzyme with the respective substrates can effect contacting.

The term “degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP>3 denotes polymers with a degree of polymerization of greater than 3.

As used herein the term “ethanol producer” or ethanol producing microorganism” refers to any organism or cell that is capable of producing ethanol from a hexose or pentose. Generally, ethanol-producing cells contain an alcohol dehydrogenase and a pyruvate decarboxylase. Examples of ethanol producing microorganisms include fungal microorganisms such as yeast. A preferred yeast includes strains of Saccharomyces, particularly, S. cerevisiae.

“Fermentation” is the enzymatic and anaerobic breakdown of organic substances by microorganisms to produce simpler organic compounds. While fermentation occurs under anaerobic conditions, it is not intended that the term be solely limited to strict anaerobic conditions, as fermentation also occurs in the presence of oxygen.

The term “heterologous” with reference to a polynucleotide or protein refers to a polynucleotide or protein that does not naturally occur in a host cell.

As used herein the term “saccharification” refers to enzymatic conversion of complex carbohydrates (e.g., cellulose, starch) to simple sugars (e.g., glucose).

As used herein, the term “starch” refers to a complex polysaccharide of plants comprising amylose and amylopectin with the formula (C₆H₁₀O₅)ₓ, wherein “X” can be any number. Examples of plant-based materials comprising starch include but are not limited to grains, grasses, tubers and roots and more specifically wheat, barley, maize, rye, rice, sorghum, brans, cassava (tapioca), millet, potato, and sweet potato.

By “thermostable” is meant the ability of an enzyme to retain activity after exposure to elevated temperatures.

The term “yield” refers to the amount of end-product or desired end-products produced using the methods described herein. In certain embodiments, the yield is greater than that produced using methods known in the art. In some embodiments, the term refers to the volume of the end product and in other embodiments the term refers to the concentration of the end product.

The methods of the disclosure involve the conversion of cellulose in grain to glucose that can then be used in the production of ethanol or other desired molecules via fermentation methods known in the art. To convert the cellulose to glucose, the methods of the disclosure involve contacting the grain with one or more cellulases. In certain embodiments, the methods of the disclosure involve producing in the grain one or more heterologous cellulases.

For the degradation of cellulose, two general types of cellulases can be employed. Cellulases which cleave the cellulose chain internally are referred to as endoglucanases (E.C. 3.2.1.4) and serve to provide new reducing and non-reducing chain termini on which cellobiohydrolases (E.C. 3.2.1.91) can operate. The product of the cellobiohydrolase is typically cellobiose, so a third activity, β-glucosidase (E.C. 3.2.1.21), is required to cleave cellobiose to glucose. The cellobiohydrolase can also yield longer glucose chains (up to 6 glucose units) that require a β-glucosidase activity to reduce their size.

Relative to the other enzyme activities needed for degradation of cellulose, only a minor amount of the β-glucosidase activity is required. Therefore, the necessary β-glucosidase activity can be supplied from β-glucosidase that is added during saccharification and/or fermentation.

There are numerous cellulase genes cloned and sequenced from a wide variety of bacteria and fungi that can be utilized in the methods of the present disclosure. For example, see, Mohagheghi et al. (1986) Isolation and Characterization of Acidothermus cellulolyticus gen. nov., sp. nov., a new genus of thermophillic, acidophillic, cellulolytic bacteria. Int. J. Syst. Bacteriol. 36:435-443; Nieves et al. (1995) Appl. Biochem. Biotechnol. 51/52:211-223; U.S. Pat. No. 5,536,655; Shoemaker et al. (1983). Molecular Cloning of Exo-Cellobiohydrolase I Derived From Trichoderma reesei Strain L27. Bio/Technology 691-696; Schulein M, 2000. Protein engineering of cellulases. Biochim. Biophys. Acta 1543:239-252; Tomme P, et al., 1995. Cellulose Hydrolysis by Bacteria and Fungi. Advances in Microbial Physiology 37:1-81; Ziegler, M T, et al. 2000, Accumulation of a thermostable endo-1,4-β-D-glucanase in the apoplast of Arabidopsis thaliana leaves. Molecular Breeding 6:37-46; Dai Z, et al. 2000. Improved plant-based production of E1 endoglucanase using potato: expression optimization and tissue targeting. Molecular Breeding 6:277-285; Ziegelhoffer T, et al. 1999. Expression of bacterial cellulase genes in transgenic alfalfa (Medicago sativa L.), potato (Solanum tuberosum L.) and tobacco (Nicotiana tabacum L.). Molecular Breeding 5:309-318; Henrissat B. A, Classification of glycosyl hydrolases based on amino-acid sequence similarities Biochem. J. 280:309-316 (1991); Henrissat B., Bairoch A., New families in the classification of glycosyl hydrolases based on amino-acid sequence similarities, Biochem. J 293:781-788 (1993); Henrissat B., Bairoch A. Updating the sequence-based classification of glycosyl hydrolases, Biochem. J. 316:695-696 (1996); Davies G., Henrissat B, Structures and mechanisms of glycosyl hydrolases, Structure 3:853-859 (1995); Jang. S. J. et al, New integration vector using a cellulase gene as a screening marker for Lactobacillus, FEMS Microbiol Lett. 2003 Jul. 29; 224 (2): 191-5; Rees, H. C. et al. Detecting cellulase and esterase enzyme activities encoded by novel genes present in environmental DNA libraries. Extremophiles. Jul. 5, 2003; Moriya, T. et al. Cloning and overexpression of the avi2 gene encoding a major cellulase produced by Humicola insolens FERM BP-5977. Biosci Biotechnol Biochem. 2003 June; 67 (6): 1434-7; Sanchez, M M et al., Exo-mode of action of cellobiohydrolase Cel48C from Paenibacillus sp. BP-23. A unique type of cellulase among Bacillales. Eur J Biochem. 2003 July; 270 (13): 2913-9; Abdeev, R. M. et al, Expression of a thermostable bacterial cellulase in transgenic tobacco plants Genetika. 2003 March; 39 (3): 376-82.; PMID: 12722638; Qin Q et al., Characterization of a tomato protein that inhibits a xyloglucan-specific endoglucanase. Plant J. 2003 May; 34 (3): 327-38.; Murray P. G. et al., Molecular cloning, transcriptional, and expression analysis of the first cellulase gene (cbh2), encoding cellobiohydrolase II, from the moderately thermophilic fungus Talaromyces emersonii and structure prediction of the gene product. Biochem Biophys Res Commun. 2003 Feb. 7; 301 (2): 280-6; Nakashima, K. I. et al., Cellulase genes from the parabasalian symbiont Pseudotrichonympha grassii in the hindgut of the wood-feeding termite Coptotermes formosanus. Cell Mol Life Sci. 2002 September; 59 (9): 1554-60. The above is a small sampling of the myriad of cellulase encoding genes available to one skilled in the art.

Nucleotide sequences encoding cellulases (e.g., endoglucanases and cellobiohydrolases) are known in the art. Nucleotide sequences encoding endoglucanases include, but are not limited to, the nucleotide sequence having GenBank Accession No. U33212. Nucleotide sequences encoding cellobiohydrolases include, but are not limited to, the nucleotide sequence having GenBank Accession No. X69976.

A cellulase may be derived from any microbial source, such as a bacterium or fungus. Many microorganisms make enzymes that hydrolyze cellulose, including the bacteria such as Acidothermus, Thermobifida, Bacillus, Cellulomonas, Clostridium, Pseudomonas, and Streptomyces; and fungi such as Acremonium, Aspergillus, Aureobasidium, Chrysosporium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, and Trichoderma. In certain embodiments, the methods can involve, one, two, three, four, five, or more of such cellulases.

In certain embodiments, the cellulase comprises endoglucanase E1 from Acidothermus cellulolyticus. In certain embodiments, the cellulase comprises cellobiohydrolase I or cellobiohydrolase II from Trichoderma reesei. In certain embodiments, the cellulase comprises endoglucanase E1 from Acidothermus cellulolyticus, cellobiohydrolase I from Trichoderma reesei, and cellobiohydrolase II from Trichoderma reesei.

Examples of cellulases (e.g., endoglucanase, cellobiohydrolase I, cellobiohydrolase II) also include the amino acid sequences set forth in SEQ ID NOs: 1, 3, and 5. Polynucleotide sequences encoding such amino acid sequences include SEQ ID NOs: 2, 4, and 6.

In certain embodiments, the polynucleotide encodes a cellulase comprising an amino acid sequence having at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity to the full length or a fragment of the amino acid sequence of SEQ ID NO: 1, 3, or 5. In certain embodiments, the polynucleotide comprises a nucleotide sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% to the full length or a fragment of the nucleotide sequence of SEQ ID NO: 2, 4, or 6.

Sequence alignments and percent identity calculations may be determined using a variety of comparison methods designed to detect similar or identical sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CAB/OS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CAB/OS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program. In certain embodiments, the % sequence identity is determined over the entire length of the molecule (nucleotide or amino acid).

Commercially available cellulases include, for example, CELLIC® CTec (Novozymes A/S), CELLIC® CTec2 (Novozymes A/S), CELLUCLAST® (Novozymes A/S), NOVOZYM™ 188 (Novozymes A/S), CELLUZYME™ (Novozymes A/S), CEREFLO™ (Novozymes A/S), and ULTRAFLO™ (Novozymes A/S), ACCELERASE™ (Genencor Int.), LAMINEX™ (Genencor Int.), SPEZYME™ CP (Genencor Int.), FILTRASE® NL (DSM); METHAPLUS® S/L 100 (DSM), ROHAMENT™ 7069 W (Röhm GmbH), FIBREZYME® LDI (Dyadic International, Inc.), FIBREZYME® LBR (Dyadic International, Inc.), VISCOSTAR® 150L (Dyadic International, Inc.), CLAZINASE® (DuPont Industrial Biosciences), and PURADAX® HA (DuPont Industrial Biosciences).

In certain embodiments, the heterologous cellulase comprises an endoglucanase and a cellobiohydrolase. The weight ratio of the endoglucanase to the cellobiohydrolase may be from about 50:1 to about 1:50, from about 25:1 to about 1:25, or from about 10:1 to about 1:10, or any value or subrange within the recited ranges, including endpoints. For example, weight ratio of the endoglucanase to the cellobiohydrolase may be about 1:1, 1:2, 1:3, 1:4, 1:5, 1:6, 1:7, 1:8, 1:9, 1:10, 1:15, 1:20, 1:25, or 1:30.

In certain embodiments, the cellulase is sufficiently thermostable to withstand the heat treatment required for liquefaction of starch. In certain embodiments, the cellulase is thermostable to at least 80° C., at least 85° C., at least 90° C., at least 95° C., at least 100° C., or at least 105° C. In a broad sense, thermostable enzymes can be derived from thermophilic microorganisms. They can also be further improved, modified, synthesized, or recombined through any known molecular biology and protein engineering tools beyond the wild type. These tools include both random mutations and site-specific mutations.

One or more of a hemicellulase, mannanase, xylanase, pectinase, or other cell wall active enzyme may be used as an additional enzyme in the present compositions and methods. In certain other embodiments, the composition is free or substantially free of other cell wall active enzymes. For example, the composition may be free or substantially free of a hemicellulase, mannanase, and/or xylanase.

One reason that cellulose utilization has not yet been commercially realized is due to the high cost of the cellulases required. The methods of the present disclosure provide for the cost-effective production of cellulases in grain, particularly transgenic maize grain. However, the methods of the disclosure find use with any plant species capable of producing a heterologous cellulase. In certain embodiments, the cellulases are produced in a particular portion of the grain, such as, for example, in the embryo, endosperm, seed coat, bran, or hull.

In general ethanol production from grain can be separated into four main steps: (1) milling, (2) liquefaction, (3) saccharification, and (4) fermentation.

The grain is milled in order to open up the structure and allow for further processing. Two processes used are wet or dry milling. In dry milling, the whole kernel is milled and used in the remaining part of the process. Wet milling gives a very good separation of germ and meal (starch granules and protein) and is, with a few exceptions, applied at locations where there is a parallel production of syrups.

In the liquefaction process, the starch granules are solubilized by hydrolysis to maltodextrins mostly of a DP higher than 4. The hydrolysis may be carried out by acid treatment or enzymatically by amylases. The amylase typically used for this application is a thermally stable, bacterial α-amylase, such as a Geobacillus stearothermophilus α-amylase. Acid hydrolysis is used on a limited basis. The raw material can be milled whole grain or a side stream from starch processing.

Enzymatic liquefaction is typically carried out as a three-step hot slurry process. The slurry is heated to between about 60° C. and about 95° C., typically about 80° C. to about 85° C., and the enzyme(s) is (are) added. Then the slurry is jet-cooked at between about 95° C. to about 140° C., typically about 105° C. to about 125° C., cooled to between about 60° C. to about 95° C. and more enzyme(s) is (are) added to obtain the final hydrolysis. The liquefaction process is carried out at a pH between 4.5 and 6.5, typically at a pH between 5 and 6. Milled and liquefied grain is also known as mash.

To produce low molecular sugars DP₁₋₃ that can be metabolized by yeast, the maltodextrin from the liquefaction must be further hydrolyzed. The hydrolysis is typically done enzymatically by glucoamylases, alternatively α-glucosidases or acid stable α-amylases can be used. A full saccharification step may last up to 72 hours, however, it is common only to do a pre-saccharification of typically between 40 and 90 minutes and then complete saccharification during fermentation (SSF). Saccharification is typically carried out at temperatures from 30° C. to 65° C., typically around 60° C., and at pH 4.5.

Yeast typically from Saccharomyces spp. is added to the mash and the fermentation is ongoing for 24 to 96 hours, typically 35 to 60 hours. The temperature is between 26° C. and 34° C., typically at about 32° C., and the pH is from 3 to 6, typically pH 4 to 5.

Note that the most widely used process is a simultaneous saccharification and fermentation (SSF) process where there is no holding stage for the saccharification, meaning that yeast and enzyme are added together. When doing SSF it is common to introduce a pre-saccharification step at a temperature above 50° C., just prior to the fermentation.

According to a method described herein, the saccharification and fermentation may be carried out simultaneously or separately.

Following the fermentation, the mash is typically distilled to extract the ethanol. The ethanol obtained according to the process described herein may be used as, e.g., fuel ethanol; drinking ethanol, i.e., potable neutral spirits; or industrial ethanol.

Left over from the fermentation is the grain, which is typically used for animal feed cither in liquid form or dried.

Further details on how to carry out liquefaction, saccharification, fermentation, distillation, and recovery of ethanol are well known to the skilled person.

By combining saccharification of cellulose in the grain with that of starch, the glucose yield (and thus the ethanol yield) is significantly increased compared to saccharification of starch alone. In certain embodiments, the glucose yield (and optionally the ethanol yield) is increased at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% relative to a method without a cellulase. In certain embodiments, the methods of the disclosure provide increased glucose yield (and optionally the ethanol yield) relative to a method wherein the milled grain does not comprise a heterologous cellulase.

Applicants have found that milled grain comprising the heterologous cellulase (e.g., an endoglucanase and/or a cellobiohydrolase) can be combined with control milled grain (i.e., milled grain without the heterologous cellulase) and yield similar amounts of glucose. In certain embodiments, from about 1% to about 95%, from about 5% to about 90%, or from about 10% to about 50% of the milled grain comprises the heterologous cellulase, or any value or subrange within the recited ranges, including endpoints. For example, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the milled grain can comprise the heterologous cellulase. In certain embodiments, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 25%, less than 20%, less than 15%, or less than 10% of the milled grain comprises the heterologous cellulase.

In certain embodiments, the milled grain comprises from about 0.1% to about 5%, from about 0.5% to about 2%, or from about 0.1% to about 1% by dry weight of the heterologous cellulase, or any value or subrange within the recited ranges, including endpoints. For example, the milled grain can comprise at least about 0.05%, at least about 0.06%, at least about 0.07%, at least about 0.08%, at least about 0.09%, at least about 0.1%, at least about 0.11%, at least about 0.12%, at least about 0.13%, at least about 0.14%, at least about 0.15%, at least about 0.16%, at least about 0.17%, at least about 0.18%, at least about 0.19%, at least about 0.2%, at least about 0.3%, at least about 0.4%, at least about 0.5%, or at least about 0.6% by dry weight of the heterologous cellulase.

In certain embodiments, high expression levels of at least 10 mg/kg of whole grain are obtained. An embodiment provides for a range of about 10 mg/kg to 10 g/kg. Another embodiment provides for a range of about 1 g/kg to about 6 g/kg. Further embodiments provide for expression levels of at least 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 50 mg/kg, 60 mg/kg, 70 mg/kg, 80 mg/kg, 90 mg/kg, 100 mg/kg, 150 mg/kg, 200 mg/kg, 500 mg/kg, 1 g/kg, 2 g/kg, 3 g/kg, 4 g/kg, or 5 g/kg of whole grain or more or amounts in-between. In certain embodiments, the heterologous cellulase is expressed in the grain at levels of at least about 0.1% total soluble protein, at least about 1% total soluble protein, at least about 10% total soluble protein, or at least about 20% total soluble protein.

A variety of assays for cellulase are known in the art which can be used to detect enzyme activity in seeds of plants having the heterologous protein. See, Coughlan et al. ((1988) J. Biol. Chem. 263:16631-16636) and Freer ((1993) J. Biol. Chem. 268:9337-9342). In addition, western analysis and ELISAs can be used to assess protein integrity and expression levels.

A western analysis is a variation of the Southern analysis technique. With a Southern analysis, DNA is cut with restriction endonucleases and fractionated on an agarose gel to separate the DNA by molecular weight and then transferring to nylon membranes. It is then hybridized with the probe fragment which was radioactively labeled with ³²P and washed in an SDS solution. In the western analysis, instead of isolating DNA, the protein of interest is extracted and placed on an acrylamide gel. The protein is then blotted onto a membrane and contacted with a labeling substance. See e.g., Hood et al., “Commercial Production of Avidin from Transgenic Maize; Characterization of Transformants, Production, Processing, Extraction and Purification” Molecular Breeding 3:291-306 (1997).

The ELISA or enzyme linked immunoassay has been known since 1971. In general, antigens solubilized in a buffer are coated on a plastic surface. When serum is added, antibodies can attach to the antigen on the solid phase. The presence or absence of these antibodies can be demonstrated when conjugated to an enzyme. Adding the appropriate substrate will detect the amount of bound conjugate which can be quantified. A common ELISA assay is one which uses biotinylated anti-(protein) polyclonal antibodies and an alkaline phosphatase conjugate. For example, an ELISA used for quantitative determination of laccase levels can be an antibody sandwich assay, which utilizes polyclonal rabbit antibodies obtained commercially. The antibody is conjugated to alkaline phosphatases for detection. In another example, an ELISA assay to detect trypsin or trypsinogen uses biotinylated anti-trypsin or anti-trypsinogen polyclonal antibodies and a streptavidin-alkaline phosphatase conjugate.

The use of the term “polynucleotide” is not intended to limit a polynucleotide of the disclosure to a polynucleotide comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the disclosure also encompass all forms of sequences including, but not limited to, single-stranded forms, double-stranded forms, hairpins, stem-and-loop structures, and the like.

For example, a polynucleotide construct may be a recombinant DNA construct. A “recombinant DNA construct” comprises two or more operably linked DNA segments which are not found operably linked in nature. Non-limiting examples of recombinant DNA constructs include a polynucleotide of interest or active variant or fragment thereof operably linked to heterologous sequences which aid in the expression, autologous replication, and/or genomic insertion of the sequence of interest. Such heterologous and operably linked sequences include, for example, promoters, termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

The cellulase polynucleotides disclosed herein can be provided in expression cassettes for expression in the plant of interest or any organism of interest. The cassette can include 5′ and 3′ regulatory sequences operably linked to a cellulase polynucleotide or active variant or fragment thereof. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the cellulase polynucleotide or active variant or fragment thereof to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a cellulase polynucleotide or active variant or fragment thereof, and a transcriptional and translational termination region (i.e., termination region) functional in plants. The regulatory regions (i.e., promoters, transcriptional regulatory regions, and translational termination regions) and/or the cellulase polynucleotide or active variant or fragment thereof may be native/analogous to the host cell or to each other. Alternatively, the regulatory regions and/or the cellulase polynucleotide of or active variant or fragment thereof may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region or active variant or fragment thereof, may be native with the plant host, or may be derived from another source (i.e., foreign or heterologous) to the promoter, the cellulase polynucleotide or active fragment or variant thereof, the plant host, or any combination thereof.

The expression cassettes may additionally contain 5′ leader sequences. Such leader sequences can act to enhance translation. Translation leaders are known in the art and include viral translational leader sequences.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used to express the various cellulase sequences disclosed herein, including the native promoter of the polynucleotide sequence of interest. The promoters can be selected based on the desired outcome. Such promoters include, for example, constitutive, inducible, tissue-preferred, or other promoters for expression in plants or in any organism of interest.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

In certain embodiments, the promoter is a seed-preferred promoter that is active during seed development. For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, globulin 1, etc. Seed-preferred promoters of particular interest are those promoters that direct gene expression predominantly to specific tissues within the seed such as, for example, the endosperm-preferred promoter of γ-zein, the cryptic promoter from tobacco (Fobert et al. 1994). T-DNA tagging of a seed coat-specific cryptic promoter in tobacco. Plant J. 4:567-577), the P-gene promoter from corn (Chopra et al. 1996. Alleles of the maize P gene with distinct tissue specificities encode Myb-homologous proteins with C-terminal replacements. Plant Cell 7:1149-1158, Erratum in Plant Cell. 1997, 1:109), the globulin-1 promoter from corn (Belanger and Kriz. 1991. Molecular basis for Allelic Polymorphism of the maize Globulin-1 gene. Genetics 129:863-972), and promoters that direct expression to the seed coat or hull of corn kernels, for example the pericarp-specific glutamine synthetase promoter (Muhitch et al., 2002. Isolation of a Promoter Sequence From the Glutamine Synthetase₁₋₂ Gene Capable of Conferring Tissue-Specific Gene Expression in Transgenic Maize. Plant Science 163:865-872); Genbank accession number AF359511.

In certain embodiments, the maize globulin promoter (PGNpr25) is used. This is the promoter of the maize globulin-1 gene, described by Belanger, F. C. and Kriz, A. L. 1991. Molecular Basis for Allelic Polymorphism of the maize Globulin-1 gene. Genetics 129:863-972. It also can be found as accession number L22344 in the Genbank database. Another example is the phaseolin promoter. See, Bustos et al. 1989. Regulation of B-glucuronidase expression in transgenic tobacco plants by an A/T-rich cis-acting sequence found upstream of a french bean B-phaseolin gene. The Plant Cell. (1): 839-853. In certain embodiments, the rice glutelin promoter or the rice globulin promoter is used. See, Vicuna Requesens, et al., In Vitro Cell. Dev. Biol.—Plant, 46:485-490 (2010).

Synthetic promoters can be used to express cellulase sequences or biologically active variants and fragments thereof. Synthetic promoters include for example a combination of one or more heterologous regulatory elements.

In certain embodiments, the expression cassettes also contain a signal sequence located between the promoter and the cellulase sequences or biologically active variants and fragments thereof. A signal sequence is a nucleotide sequence, translated to give an amino acid sequence, which is used by a cell to direct the protein or polypeptide of interest to be placed in a particular place within or outside the eukaryotic cell. Many signal sequences are known in the art. See, for example Becker et al., Plant Mol. Biol. 20:49 (1992), Close, P. S., Master's Thesis, Iowa State University (1993), Knox, C., et al., “Structure and Organization of Two Divergent Alpha-Amylase Genes from Barley”, Plant Mol. Biol. 9:3-17 (1987), Lemer et al., Plant Physiol. 91:124-129 (1989), Fontes et al., Plant Cell 3:483-496 (1991), Matsuoka et al., Proc. Natl. Acad. Sci. 88:834 (1991), Gould et al., J. Cell. Biol. 108:1657 (1989), Creissen et al., Plant J. 2:129 (1991), Kalderon, et al., A short amino acid sequence able to specify nuclear location, Cell 39:499-509 (1984), Steifel, et al., Expression of a maize cell wall hydroxyproline-rich glycoprotein gene in early leaf and root vascular differentiation, Plant Cell 2:785-793 (1990). When targeting the enzyme to the cell wall use of a signal sequence is necessary. One example is the barley alpha-amylase signal sequence (Rogers, J. C. 1985. Two barley alpha-amylase gene families are regulated differently in aleurone cells. J. Biol. Chem. 260:3731-3738).

In certain embodiments, the enzyme production is retained in the endoplasmic reticulum of the plant cell. This may be accomplished by use of a localization sequence, such as KDEL, which contains the binding site for a receptor in the endoplasmic reticulum. (Munro, S. and Pelham, H. R. B. 1987 “A C-terminal signal prevents secretion of luminal ER proteins” Cell 48:899-907. The use of such a localization sequence will increase expression over levels obtained when the enzyme is otherwise expressed in the cytoplasm.

In certain embodiments, the cellulase is targeted to the vacuole. Signal sequences to accomplish this are well known. For example, Raikhel U.S. Pat. No. 5,360,726 shows a vacuole signal sequence as does Warren et al at U.S. Pat. No. 5,889,174. Vacuolar targeting signals may be present either at the amino-terminal portion, (Holwerda et al., The Plant Cell, 4:307-318 (1992), Nakamura et al., Plant Physiol., 101:1-5 (1993)), carboxy-terminal portion, or in the internal sequence of the targeted protein. (Tague et al., The Plant Cell, 4:307-318 (1992), Saalbach et al. The Plant Cell, 3:695-708 (1991)). Additionally, amino-terminal sequences in conjunction with carboxy-terminal sequences are responsible for vacuolar targeting of gene products (Shinshi et al. Plant Molec. Biol. 14:357-368 (1990)).

Various methods can be used to introduce a sequence of interest into a host cell, plant or plant part. “Introducing” is intended to mean presenting to the host cell, plant, plant cell or plant part the polynucleotide or polypeptide in such a manner that the sequence gains access to the interior of a cell of the plant or organism. The methods of the disclosure do not depend on a particular method for introducing a sequence into an organism or a plant or plant part, only that the polynucleotide or polypeptides gains access to the interior of at least one cell of the organism or the plant. Methods for introducing polynucleotide or polypeptides into various organisms, including plants, are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and biolistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaccac); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In certain embodiments, the cellulase sequences or active variants or fragments thereof can be provided to a plant using a variety of transient transformation methods. Such transient transformation methods include, but are not limited to, the introduction of the cellulase or active variants and fragments thereof directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91:2176-2180 and Hush et al. (1994) The Journal of Cell Science 107:775-784, all of which are herein incorporated by reference.

Methods are known in the art for the targeted insertion of a polynucleotide at a specific location in the plant genome. As an example, the plant described herein, is generated using “custom” or engineered endonucleases such as meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11 (9): 636-46; Shukla, et al., (2009) Nature 459 (7245): 437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39 (12) and Boch et al., (2009), Science 326 (5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9:39; The CRISPR/Cas system allows targeted cleavage of genomic DNA guided by a customizable small noncoding RNA.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide disclosed herein, for example, as part of an expression cassette, stably incorporated into their genome.

Transformed plant cells which are derived by plant transformation techniques, including those discussed above, can be cultured to regenerate a whole plant which possesses the transformed genotype (i.e., a cellulase polynucleotide), and thus the desired phenotype. For transformation and regeneration of maize see, Gordon-Kamm et al., The Plant Cell, 2:603-618 (1990). Plant regeneration from cultured protoplasts is described in Evans et al. (1983) Protoplasts Isolation and Culture, Handbook of Plant Cell Culture, pp 124-176, Macmillan Publishing Company, New York; and Binding (1985) Regeneration of Plants, Plant Protoplasts pp 21-73, CRC Press, Boca Raton. Regeneration can also be obtained from plant callus, explants, organs, or parts thereof. Such regeneration techniques are described generally in Klee et al. (1987) Ann Rev of Plant Phys 38:467. See also, e.g., Payne and Gamborg.

One of skill will recognize that after the expression cassette containing the cellulase gene is stably incorporated in transgenic plants and confirmed to be operable, it can be introduced into other plants by sexual crossing. Any of a number of standard breeding techniques can be used, depending upon the species to be crossed.

In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that would produce the selected phenotype.

Parts obtained from the regenerated plant, such as flowers, seeds, leaves, branches, fruit, and the like are included, provided that these parts comprise cells comprising the cellulase nucleic acid. Progeny and variants, and mutants of the regenerated plants are also included, provided that these parts comprise the introduced nucleic acid sequences.

In certain embodiments, a homozygous transgenic plant can be obtained by sexually mating (selfing) a heterozygous transgenic plant that contains a single added heterologous nucleic acid, germinating some of the seed produced and analyzing the resulting plants produced for altered cell division relative to a control plant (i.e., native, non-transgenic). Back-crossing to a parental plant and out-crossing with a non-transgenic plant are also contemplated.

Further provided are engineered host cells that are transformed with one or more cellulase sequences or active variants or fragments thereof. The cellulase polypeptides or variants and fragments thereof can be expressed in any organism, including in non-animal cells such as plants, yeast, fungi, bacteria and the like. Details regarding non-animal cell culture can be found in Payne et al. (1992) Plant Cell and Tissue Culture in Liquid Systems, John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin, Heidelberg, N.Y.); and Atlas and Parks (eds.) The Handbook of Microbiological Media (1993) CRC Press, Boca Raton, Fla.

Plants, plant cells, plant parts and seeds having the cellulase sequences disclosed herein are also provided. In specific embodiments, the plants and/or plant parts have stably incorporated at least one heterologous cellulase polypeptide disclosed herein or an active variant or fragment thereof. In addition, the plants or organism of interest can comprise multiple cellulase polynucleotides (i.e., at least 1, 2, 3, 4, 5, 6 or more).

In certain embodiments, the heterologous cellulase polynucleotide in the plant or plant part is operably linked to a heterologous regulatory element, such as but not limited to a constitutive, tissue-preferred, or other promoter for expression in plants or a constitutive enhancer.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, cars, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

The cellulase sequences and active variants and fragments thereof disclosed herein may be used for transformation of any plant species, including, but not limited to, monocots and dicots. Examples of plant species of interest include, but are not limited to, corn (Zea mays), Brassica sp. (e.g., B. napus, B. rapa, B. juncea), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley.

A “subject plant or plant cell” is one in which genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

The following numbered embodiments also form part of the present disclosure:
1. A method of producing glucose, the method comprising: contacting milled grain comprising an effective amount of a heterologous cellulase with an enzyme composition comprising β-glucosidase under conditions sufficient to hydrolyze cellulose in the milled grain to glucose.
2. The method of embodiment 1, wherein the milled grain comprising the heterologous cellulase is from a transgenic plant comprising a heterologous polynucleotide encoding the cellulase.
3. The method of embodiment 1 or embodiment 2, wherein at least about 5% of the milled grain comprises the heterologous cellulase.
4. The method of any one of embodiments 1-3, wherein from about 10% to about 50% of the milled grain comprises the heterologous cellulase.
5. The method of any one of embodiments 1-4, wherein the milled grain comprises at least about 0.1% by dry weight of the heterologous cellulase.
6. The method of any one of embodiments 1-5, wherein the heterologous cellulase comprises an endoglucanase and/or a cellobiohydrolase.
7. The method of any one of embodiments 1-6, wherein the heterologous cellulase is thermostable.
8. The method of any one of embodiments 1-7, wherein the heterologous cellulase is from a genus selected from Acidothermus, Acremonium, Aspergillus, Aureobasidium, Bacillus, Cellulomonas, Chrysosporium, Clostridium, Cryptococcus, Filibasidium, Fusarium, Humicola, Magnaporthe, Mucor, Myceliophthora, Neocallimastix, Neurospora, Paecilomyces, Penicillium, Piromyces, Pseudomonas, Schizophyllum, Streptomyces; Talaromyces, Thermoascus, Thermobifida, Thielavia, Tolypocladium, and Trichoderma.
9. The method of any one of embodiments 1-8, wherein the heterologous cellulase comprises endoglucanase E1 from Acidothermus cellulolyticus, cellobiohydrolase I from Trichoderma reesei, and/or cellobiohydrolase II from Trichoderma reesei.
10. The method of any one of embodiments 1-9, wherein the milled grain is contacted with the enzyme composition at a temperature of about 20° C., 30° C., 40° C., or 45° C. to about 55° C., 60° C., 70° C., or 80° C., optionally wherein the temperature is about 50° C.
11. The method of any one of embodiments 1-10, wherein the milled grain is contacted with the enzyme composition for at least about 12 hours, about 16 hours, or about 24 hours.
12. The method of any one of embodiments 1-11, further comprising heating the milled grain prior to contacting.
13. The method of embodiment 12, wherein the milled grain is heated at a temperature of about 50° C., 60° C., 70° C., or 80° C. to about 90° C. or 100° C., optionally wherein the temperature is about 85° C.
14. The method of embodiment 12 or embodiment 13, wherein the milled grain is heated for about 30 seconds, 1 minute, or 2 minutes to about 10 minutes, 30 minutes, or 1 hour, optionally wherein the milled grain is heated for about 5 minutes.
15. The method of any one of embodiments 1-14, further comprising contacting the milled grain with one or more amylases under conditions sufficient to hydrolyze starch in the milled grain to glucose.
16. The method of any one of embodiments 1-15, wherein the one or more amylases comprise an α-amylase and amyloglucosidase.
17. The method of any one of embodiments 1-16, further comprising incubating the milled grain for a time and at a temperature sufficient to liquefy the starch.
18. The method of embodiment 17, wherein the temperature is about 85° C. to about 100° C.
19. The method of any one of embodiments 1-18, wherein the milled grain is maize, wheat, rice, or sorghum grain.
20. The method of any one of embodiments 1-19, wherein glucose yield is increased at least 2%, at least 3%, at least 4%, at least 5%, at least 10%, or at least 15% relative to a method wherein the milled grain does not comprise a heterologous cellulase.
21. The method of any one of embodiments 1-20, further comprising incubating the glucose with at least one fermenting microorganism under conditions in which ethanol is produced.
22. A method of producing ethanol, the method comprising: contacting milled grain comprising an effective amount of a heterologous cellulase with an enzyme composition comprising β-glucosidase and one or more amylases under conditions sufficient to hydrolyze cellulose and starch in the milled grain to glucose; and incubating the glucose with at least one fermenting microorganism under conditions in which ethanol is produced.
23. The method of embodiment 22, wherein the milled grain comprising the heterologous cellulase is from a transgenic plant comprising a heterologous polynucleotide encoding the cellulase.
24. The method of embodiment 22 or embodiment 23, wherein at least about 5% of the milled grain comprises the heterologous cellulase.
25. The method of any one of embodiments 22-24, wherein from about 10% to about 50% of the milled grain comprises the heterologous cellulase.
26. The method of any one of embodiments 22-25, wherein the milled grain comprises at least about 0.1% by dry weight of the heterologous cellulase.
27. The method of any one of embodiments 22-26, wherein the heterologous cellulase comprises an endoglucanase and/or a cellobiohydrolase.
28. The method of any one of embodiments 22-27, wherein the heterologous cellulase is thermostable.
29. The method of any one of embodiments 22-28, wherein the heterologous cellulase is from a genus selected from Acidothermus, Acremonium, Aspergillus, Aureobasidium, Bacillus, Cellulomonas, Chrysosporium, Clostridium, Cryptococcus, Filibasidium, Fusarium, Humicola, Magnaporthe, Mucor, Myceliophthora, Neocallimastix, Neurospora, Paecilomyces, Penicillium, Piromyces, Pseudomonas, Schizophyllum, Streptomyces; Talaromyces, Thermoascus, Thermobifida, Thielavia, Tolypocladium, and Trichoderma.
30. The method of any one of embodiments 22-29, wherein the heterologous cellulase comprises endoglucanase E1 from Acidothermus cellulolyticus, cellobiohydrolase I from Trichoderma reesei, and/or cellobiohydrolase II from Trichoderma reesei.
31. The method of any one of embodiments 22-30, wherein the milled grain is contacted with the enzyme composition at a temperature of about 20° C., 30° C., 40° C., or 45° C. to about 55° C., 60° C., 70° C., or 80° C., optionally wherein the temperature is about 50° C.
32. The method of any one of embodiments 22-31, wherein the milled grain is contacted with the enzyme composition for at least about 12 hours, about 16 hours, or about 24 hours.
33. The method of any one of embodiments 22-32, further comprising heating the milled grain prior to contacting.
34. The method of embodiment 33, wherein the milled grain is heated at a temperature of about 50° C., 60° C., 70° C., or 80° C. to about 90° C. or 100° C., optionally wherein the temperature is about 85° C.
35. The method of embodiment 33 or embodiment 34, wherein the milled grain is heated for about 30 seconds, 1 minute, or 2 minutes to about 10 minutes, 30 minutes, or 1 hour, optionally wherein the milled grain is heated for about 5 minutes.
36. The method of any one of embodiments 22-35, wherein the one or more amylases comprise an α-amylase and amyloglucosidase.
37. The method of any one of embodiments 22-36, further comprising incubating the milled grain for a time and at a temperature sufficient to liquefy the starch.
38. The method of embodiment 37, wherein the temperature is about 85° C. to about 100° C.
39. The method of any one of embodiments 33-38, wherein the milled grain is maize, wheat, rice, or sorghum grain.
40. The method of any one of embodiments 22-39, wherein ethanol yield is increased at least 2%, at least 3%, at least 4%, at least 5%, at least 10%, or at least 15% relative to a method wherein the milled grain does not comprise a heterologous cellulase.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended embodiments.

The following examples are offered by way of illustration and not by way of limitation.

### EXAMPLES

### Example 1

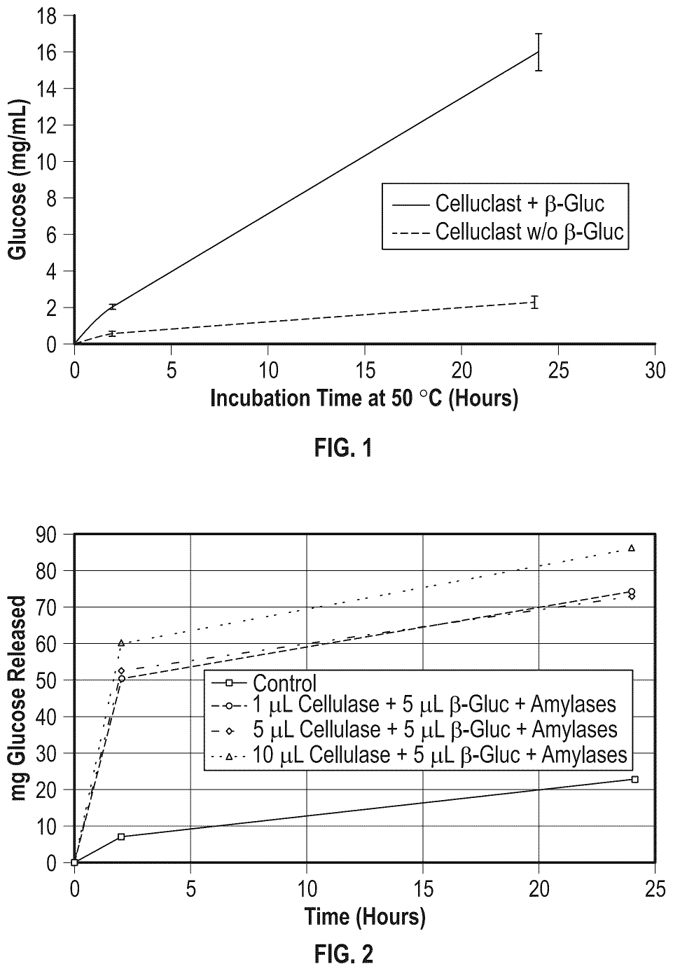

To demonstrate glucose release from cellulose in addition to that from starch, a commercial cellulase (CELLUCLAST®) was added prior to adding amylases for the starch. The flour mixtures were incubated for 24 hours in cellulase plus and minus β-glucosidase (FIG. 1). The β-glucosidase is required for the release of glucose from cellobiose.

The experiment was repeated with amylases. The cellulase and β-glucosidase were added overnight prior to heating the flour to 85° C. for 10 minutes to liquify starch. α-Amylase and amyloglucosidase were added at time zero (FIG. 2). The commercial cellulase with β-glucosidase released approximately 20 mg of glucose from the flour sample without any added amylases. When the amylases were added, up to 85 mg of glucose were released with 10 μL of commercial cellulase. One and 5 L of cellulase released significantly less (approximately 72 mg). These results demonstrate that cellulases can release more glucose from ground corn flour that could be used for fermentation into ethanol.

### Example 2

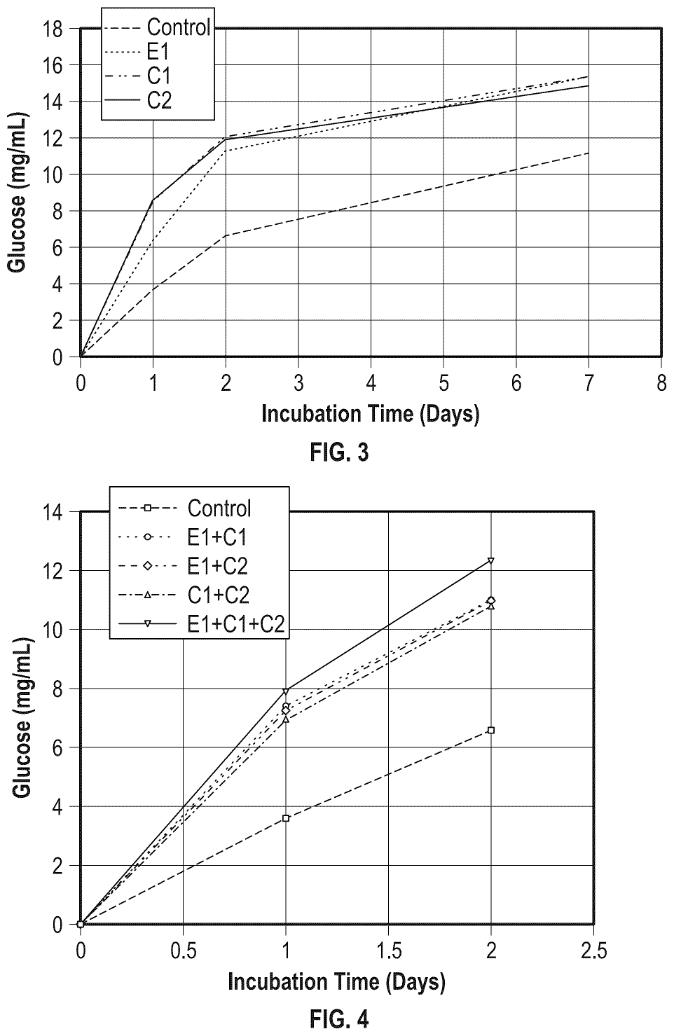

To lower the cost of cellulose digestion to improve glucose recovery, flour was made from corn that expresses different cellulases: endoglucanase E1 from Acidothermus cellulolyticus or cellobiohydrolase I (CBH I) and the cellobiohydrolase II (CBH II) from Trichoderma reesei. Corn flour containing each of the enzymes separately was incubated for 1-7 days with and without β-glucosidase. Each enzyme alone yielded low amounts of glucose, around 2%. However, when β-glucosidase was added, the yields increased dramatically to approximately 15% of the total dry weight (FIG. 3). Control flour with only β-glucosidase in this case increased 11%.

The enzymes were combined to determine if synergy was apparent when more than a single cellulase was combined with β-glucosidase (FIG. 4). After 2 days, all combinations showed 11-12% degradation with β-glucosidase whereas control flour with only β-glucosidase showed 6.5%.

### Example 3

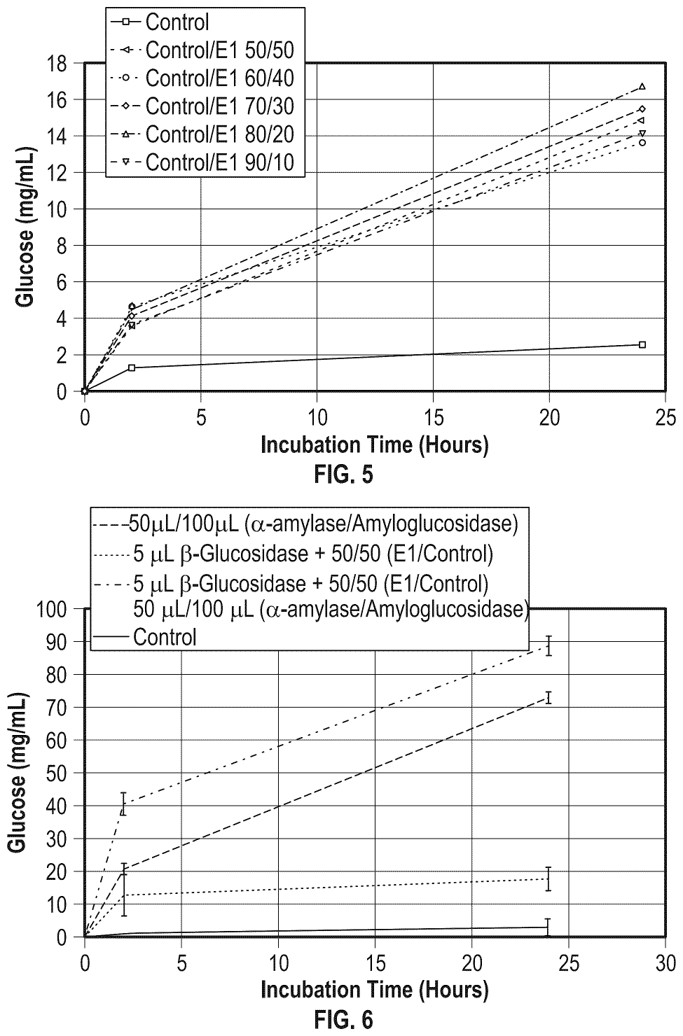

In this example, whether volumes of cellulase containing flour could be combined with control flour to produce similar amounts of glucose was explored. Flour comprising the heterologous endoglucanase E1 from Acidothermus cellulolyticus (“E1 four”) was combined with control flour in several ratios to determine how much endoglucanase E1 is required to degrade cellulose in larger volumes of flour. The ratios were 50:50 E1 flour to control flour, then 40:60; 30:70; 20:80; and 10:90 (FIG. 5). In all cases, the flour produced increased amounts of glucose, without significant differences among the ratios, although the 70:30 and 80:20 ratios were the best. These combinations of control flour and E1 flour are not what drives this combination ratio—it is the actual content of the endoglucanase E1 in the flour. The concentration of enzyme in this flour is 0.135% of dry weight.

### Example 4

Whether cellulase digestion could be combined with amylase digestion to improve recovery of glucose significantly above amylase alone was investigated. To effectively digest amylose and amylopectin (collectively starch), the starch substrate must be liquified to make the bonds available to the enzymes α-amylase and amyloglucosidase. These two enzymes effectively remove all the starch from the flour (60-70%). The E1 flour (50:50 with control flour) was incubated with β-glucosidase for 15-18 hours, then heated the mixture to 85° C. for 10 minutes to liquify the starch, finally adding the amylases for 24-hour digests (FIG. 6). The amount of glucose released with E1 flour is 10-15% higher than with the amylases alone.

### Example 5

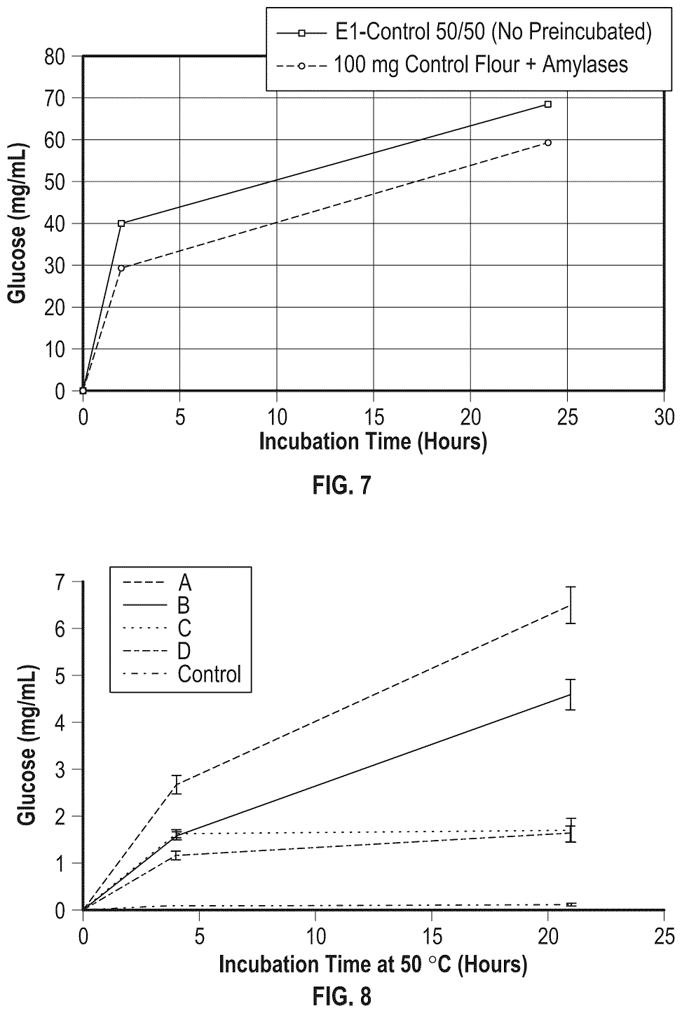

In this example, whether the E1 flour could withstand the 85° C. heat treatment of the starch and still be functional was investigated. The endoglucanase E1 is from a heat resistant hot springs bacterium, Acidothermus cellulolyticus, and is stable up to 81° C. A 90° C. treatment for 5-10 minutes severely curtails the enzyme activity. However, the 85° C. treatment for 10 minutes appeared to allow the E1 to be active, allowing a 10% increase in glucose over amylase treatment alone (FIG. 7).

### Materials and Methods for Examples 1-5

### Materials

Sodium acetate for buffering reactions and glucoamylase, CELLUCLAST® (C2730) and Novo188 β-glucosidase were purchased from Millipore Sigma (St. Louis, MO). Alpha amylase (ICN10044725) and the glucose oxidase kit (using Amplex red detection: INVITROGEN™ A22189) for glucose determination were purchased from Fisher Scientific.

### Corn Sources

The endoglucanase E1 gene from Acidothermus cellulolyticus under control of the maize globulin promoter was transformed into maize as described (Egelkrout et al., 2013; Hood et al., 2007b). The E1 construct also includes a vacuole targeting sequence. The enzyme accumulates in the apoplast of the embryo of kernels. The cellobiohydrolase I (CBHI) gene and the cellobiohydrolase II (CBHII) gene from Trichoderma reesei were transformed into corn as well (Devaiah et al., 2013; Hood et al., 2007a; Hood et al., 2014). The CBHI gene and CBHII gene are under control of the maize globulin promoter and the rice glutelin promoter, respectively. The CBHI and CBHII constructs also include the barley alpha amylase signal sequence (BAASS) for cell wall localization.

Control corn flour was produced from a Stine hybrid that is the genetic background of the E1 and CBH II transgenic lines.

### Preparing Flour Samples for Glucose Release

Corn grain was ground into flour using a coffee grinder to approximately 20 mesh size. Four types of flour were used—control and each of the 3 cellulase-containing flours described above. Flour samples (100 mg per reaction) were blended in various combinations with 1 mL of 50 mM sodium acetate buffer pH 5.0 in 1.5 mL capped tubes. Five μL β-glucosidase (Novo188) was added where indicated. Control cellulase reactions contained CELLUCLAST®. All reactions were carried out at 50° C. and rotated end over end. Flour samples were held at 85° C. for 10 minutes prior to adding the starch degrading enzymes. Five μL samples were withdrawn at the indicated sampling times. The glucose oxidase assay was used to quantify glucose release.

### Example 6

This example describes the identification of a new source of β-glucosidase and tested the importance of heat and/or xylanase.

The β-glucosidase used in the above examples was termed Novo188 and was obtained from Sigma-Aldrich Chemical Co. (St. Louis, MO). This preparation was no longer available and thus a new source of the enzyme was required. Two other sources of β-glucosidase were tested, Creative Enzymes (C. E.) and Creative Biogene (C. B.). The importance of heat and/or xylanase were also tested. The amounts of enzymes added to S411 X SO25 control flour is summarized in Table 1.

TABLE 1

| ID | A | B | C | D | Control |
|---|---|---|---|---|---|
| Ratio | 1:5:5 | 1:5:5 | 1:5:5 | 1:5:5 | |
| E1 μg/Units | 18 μg/0.09 U | 18 μg/0.09 U | 18 μg/0.09 U | 18 μg/0.09 U | − |
| CBHI μg/Units | 90 μg/0.012 U | 90 μg/0.012 U | 90 μg/0.012 U | 90 μg/0.012 U | − |
| CBHII | 90 μg/0.14 U | 90 μg/0.14 U | 90 μg/0.14 U | 90 μg/0.14 U | − |
| μg/Units | | | | | |
| Xylanase | X | X | X | X | − |
| B-gluc | C. E. | C. B. | C. E. | C. B. | + |
| Heat | + | + | − | − | |
| Glucose | 7.25 mg/mL | 5.0 mg/mL | 2.0 mg/mL | 2.0 mg/mL | 0 |
| Release | | | | | |

β-glucosidase from Creative Enzymes was the best source (FIG. 8). Heated samples worked better than non-heated, so it was concluded that the starch was sterically inhibiting the cellulase interaction with cellulose. Xylanase was not necessary to release glucose from the corn flour.

### Example 7

This example describes specific activity determinations for cellulases. Specific activity of each of the cellulases was determined as follows. A standard curve of 4-Methylumbelliferyl (MU) was made from 0 to 6 μM in a final volume of 200 μL that also contained 175 μL of stop buffer (0.2 M sodium carbonate, pH 11). The reactions with E1, CBH I, and CBH II contain 100 μL 50 mM sodium acetate buffer pH 5; 0.5 μg of purified enzyme protein; and 25 μL methylumbelliferyl-β-D-cellobioside (MUC; 0.02 M stock) (Sigma-Aldrich M6018). The assay was run for 30 minutes at 50° C., then read on a Biotek Synergy HT plate reader (Agilent, Winooski, VT) using fluorescence mode with 360 nm excitation and 460 nm emission and compared to the standard curve. Table 2 shows the specific activities of cellulase enzymes purified from corn.

TABLE 2

| | Specific | % Dry | g of flour |
|---|---|---|---|
| Enzyme | Activity | weight | per 100 U* |
| E1 | 5000 U/g | 0.18% | 11.12 |
| CBHI | 130 U/g | 0.56% | 137.4 |
| CBHII | 1500 U/g | 0.37% | 17.6 |

*The flour used in the following examples

### Example 8

This example describes utilizing units of activity rather than milligrams of enzyme optimal for degrading cellulose for increasing glucose for ethanol.

Previous experiments were performed using mg of enzyme rather than calculated units of activity. This was primarily because a purified CBH II was not available for specific activity determination. However, this value is now available (Duque et al., 2024). More of the exocellulases are required for cellulose degradation than of the endocellulase, E1. Therefore, higher amounts of CBH I and II to lower amounts of E1 were compared. Enzymes were added to the reaction as whole ground flour. The total enzyme amount in the reaction was 0.25 Units, enzyme flour was calculated according to specific activity and % D.W. Wild type flour (to complete 500 mg in combination with enzyme flour) was heated to 85° C. prior to the start of the reaction. The reaction volume was 5 mL of NaOAc pH=5 and it was incubated at 50° C. Each treatment is described in Table 3.

TABLE 3

| | T1 | T2 | T3 | T4 |
|---|---|---|---|---|
| Ratio | 1:20:5 | 1:20:5 | 1:2:2 | 1:2:2 |
| (E1:CBH I:CBH II) | | | | |
| E1 mg flour/Units | 1.11 mg/ | 1.11 mg/ | 5.55 mg/ | 5.55 mg/ |
| | 0.01 U | 0.01 U | 0.05 U | 0.05 U |
| CBHI mg | 275 mg/ | 275 mg/ | 137 mg/ | 137 mg/ |
| flour/Units | 0.20 U | 0.20 U | 0.10 U | 0.10 U |
| CBHII mg | 9 mg/ | 9 mg/ | 18 mg/ | 18 mg/ |
| flour/Units | 0.05 U | 0.05 U | 0.10 U | 0.10 U |
| Heat | + | − | + | − |
| Control flour | 215 mg | 215 mg | 339 mg | 339 mg |
| | (43%) | | (68%) | |

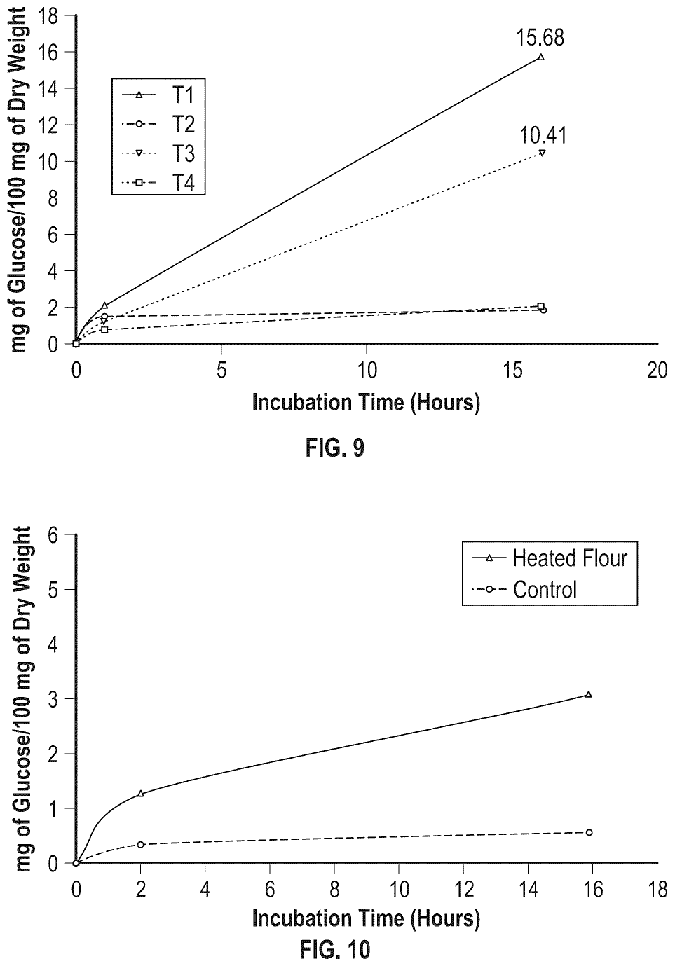

Preheating flour enabled better degradation of cellulose presumably by enabling access of the enzymes to the cellulose after liquifying starch (FIG. 9, compare T1 & T3 to T2 & T4). Higher amounts of CBH I and II digested more cellulose than lower amounts (FIG. 9, compare T1 to T3). However, lower amounts of cellulase (T3) still release enough glucose for greater profitability.

### Example 9

In this example, enzymes were heated prior to digestion holding time. The total enzyme amount in the reaction was 0.25 Units (1:20:5). Enzyme flour was calculated based on specific activity and % D.W. Control flour was added to make up a total of 500 mg. The enzyme flour, in combination with the control flour, was heated at 85° C. for 5 minutes prior to incubation. The cellulases were sensitive to heat and released 3% glucose after heating only 5 minutes (FIG. 10).

### Example 10

This example describes the digestion of cellulose in the mash that remains after saccharification and fermentation of corn grain. Mash was obtained from Tharaldson Ethanol in Casselton, North Dakota. For mash, the calculation method for the enzyme flour was based on 0.25 units (1:20:5, 285 mg) and mash was used to achieve a total of 500 mg of dry weight (1.4 mLs, where 1 mL of mash approximately equals 150 mg dry weight). In the control reaction, enzyme flour was replaced with wild type flour as a no-enzyme control, and then mash was added to reach 500 mg of dry weight. The total reaction volume was brought up to 5 mL using 50 mM NaOAc at pH=5, incubated at 50° C.

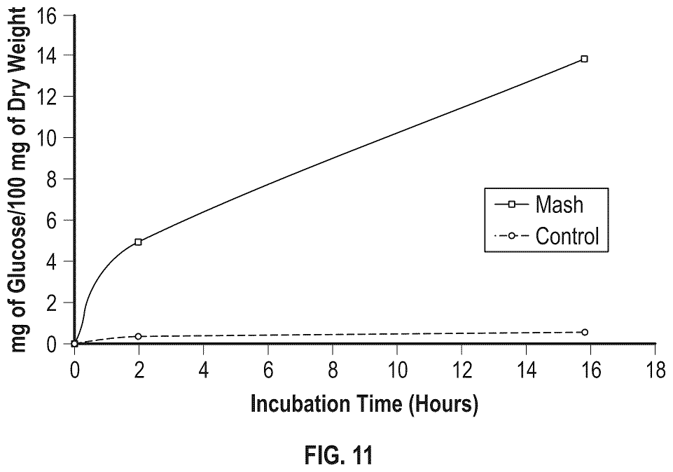

Nearly 14% of dry weight of mash was released after 16 hours incubation (FIG. 11). The mash had no remaining starch as determined by staining with iodine, so its composition was primarily cellulose and protein.

### REFERENCES

Cheng, L. and Anderson, C. (2017) Too conservative to hedge: How much does a corn ethanol facility lose? International Journal of Production Economics 193, 654-662.
Devaiah, S. P., Requesens, D. V., Chang, Y.-K., Hood, K. R., Flory, A., Howard, J. A. and Hood, E. E. (2013) Heterologous expression of cellobiohydrolase II (Cel6A) in maize endosperm. Transgenic Res 22, 477-488.
Duque, E. D. Y., Aguirre, M., Hood, N. C., & Hood, E. E. (2024). Specific activity and utility of recombinant cellobiohydrolase II (Cel6A) produced in maize endosperm. Transgenic Res, 33, 47-57.
Egelkrout, E., McGaughey, K., Keener, T., Ferleman, A., Woodard, S., Devaiah, S., Nikolov, Z., Hood, E. and Howard, J. (2013) Enhanced Expression Levels of Cellulase Enzymes Using Multiple Transcription Units. BioEnergy Research 6, 699-710.
Gombert, A. K. and van Maris, A. J. (2015) Improving conversion yield of fermentable sugars into fuel ethanol in 1st generation yeast-based production processes. Current Opinion in Biotechnology 33, 81-86.
Hood, E., Love, R., Lane, J., Bray, J., Clough, R., Pappu, K., Drees, C., Hood, K., Yoon, S. and Ahmad, A. (2007) Subcellular targeting is a key condition for high-level accumulation of cellulase protein in transgenic maize seed. Plant biotechnology journal 5, 709-719.
Hood, N.C., Hood, K. R., Woodard, S. L., Devaiah, S. P., Jeoh, T., Wilken, L., Nikolov, Z., Egelkrout, E., Howard, J. A. and Hood, E. E. (2014) Purification and Characterization of Recombinant Cel7A from Maize Seed. Applied biochemistry and biotechnology 174, 2864-2874.

What is claimed is:

1. A method of producing glucose, the method comprising:
contacting milled grain comprising an effective amount of a heterologous cellulase with an enzyme composition comprising β-glucosidase under conditions sufficient to hydrolyze cellulose in the milled grain to glucose.

2. The method of claim 1, wherein the milled grain comprising the heterologous cellulase is from a transgenic plant comprising a heterologous polynucleotide encoding the cellulase.

3. The method of claim 1, wherein at least about 5% of the milled grain comprises the heterologous cellulase.

4. The method of claim 1, wherein from about 10% to about 50% of the milled grain comprises the heterologous cellulase.

5. The method of claim 1, wherein the milled grain comprises at least about 0.1% by dry weight of the heterologous cellulase.

6. The method of claim 1, wherein the heterologous cellulase comprises an endoglucanase and/or a cellobiohydrolase.

7. The method of claim 1, wherein the heterologous cellulase is thermostable.

8. The method of claim 1, wherein the heterologous cellulase is from a genus selected from Acidothermus, Acremonium, Aspergillus, Aureobasidium, Bacillus, Cellulomonas, Chrysosporium, Clostridium, Cryptococcus, Filibasidium, Fusarium, Humicola, Magnaporthe, Mucor, Myceliophthora, Neocallimastix, Neurospora, Paecilomyces, Penicillium, Piromyces, Pseudomonas, Schizophyllum, Streptomyces; Talaromyces, Thermoascus, Thermobifida, Thielavia, Tolypocladium, and Trichoderma.

9. The method of claim 1, wherein the heterologous cellulase comprises endoglucanase E1 from Acidothermus cellulolyticus, cellobiohydrolase I from Trichoderma reesei, and/or cellobiohydrolase II from Trichoderma reesei.

10. The method of claim 1, wherein the milled grain is contacted with the enzyme composition at a temperature of about 40° C. to about 60° C.

11. The method of claim 1, wherein the milled grain is contacted with the enzyme composition for at least about 12 hours, about 16 hours, or about 24 hours.

12. The method of claim 1, further comprising heating the milled grain prior to contacting.

13. The method of claim 12, wherein the milled grain is heated at a temperature of about 70° C. to about 100° C.

14. The method of claim 12, wherein the milled grain is heated for about 30 seconds to about 10 minutes.

15. The method of claim 1, further comprising contacting the milled grain with one or more amylases under conditions sufficient to hydrolyze starch in the milled grain to glucose.

16. The method of claim 15, wherein the one more amylases comprise an α-amylase and amyloglucosidase.

17. The method of claim 15, further comprising incubating the milled grain for a time and at a temperature sufficient to liquefy the starch.

18. The method of claim 17, wherein the temperature is about 85° C. to about 100° C.

19. The method of claim 1, wherein the milled grain is maize, wheat, rice, or sorghum grain.

20. The method of claim 1, wherein glucose yield is increased at least 2%, at least 3%, at least 4%, at least 5%, at least 10%, or at least 15% relative to a method wherein the milled grain does not comprise a heterologous cellulase.

21. The method of claim 1, further comprising incubating the glucose with at least one fermenting microorganism under conditions in which ethanol is produced.

22. A method of producing ethanol, the method comprising:
contacting milled grain comprising an effective amount of a heterologous cellulase with an enzyme composition comprising β-glucosidase and one or more amylases under conditions sufficient to hydrolyze cellulose and starch in the milled grain to glucose; and incubating the glucose with at least one fermenting microorganism under conditions in which ethanol is produced.

23. The method of claim 22, wherein the milled grain comprising the heterologous cellulase is from a transgenic plant comprising a heterologous polynucleotide encoding the cellulase.

24. The method of claim 22, wherein at least about 5% of the milled grain comprises the heterologous cellulase.

25. The method of claim 22, wherein from about 10% to about 50% of the milled grain comprises the heterologous cellulase.

26. The method of claim 22, wherein the milled grain comprises at least about 0.1% by dry weight of the heterologous cellulase.

27. The method of claim 22, wherein the heterologous cellulase comprises an endoglucanase and/or a cellobiohydrolase.

28. The method of claim 22, wherein the heterologous cellulase is thermostable.

29. The method of claim 22, wherein the heterologous cellulase is from a genus selected from Acidothermus, Acremonium, Aspergillus, Aureobasidium, Bacillus, Cellulomonas, Chrysosporium, Clostridium, Cryptococcus, Filibasidium, Fusarium, Humicola, Magnaporthe, Mucor, Myceliophthora, Neocallimastix, Neurospora, Paecilomyces, Penicillium, Piromyces, Pseudomonas, Schizophyllum, Streptomyces; Talaromyces, Thermoascus, Thermobifida, Thielavia, Tolypocladium, and Trichoderma.

30. The method of claim 22, wherein the heterologous cellulase comprises endoglucanase E1 from Acidothermus cellulolyticus, cellobiohydrolase I from Trichoderma reesei, and/or cellobiohydrolase II from Trichoderma reesei.

31. The method of claim 22, wherein the milled grain is contacted with the enzyme composition at a temperature of about 40° C. to about 60° C.

32. The method of claim 22, wherein the milled grain is contacted with the enzyme composition for at least about 12 hours, about 16 hours, or about 24 hours.

33. The method of claim 22, further comprising heating the milled grain prior to contacting.

34. The method of claim 33, wherein the milled grain is heated to a temperature of about 70° C. to about 100° C.

35. The method of claim 33, wherein the milled grain is heated for about 30 seconds to about 10 minutes.

36. The method of claim 22, wherein the one more amylases comprise an α-amylase and amyloglucosidase.

37. The method of claim 22, further comprising incubating the milled grain for a time and at a temperature sufficient to liquefy the starch.

38. The method of claim 37, wherein the temperature is about 85° C. to about 100° C.

39. The method of claim 22, wherein the milled grain is maize, wheat, rice, or sorghum grain.

40. The method of claim 22, wherein ethanol yield is increased at least 2%, at least 3%, at least 4%, at least 5%, at least 10%, or at least 15% relative to a method wherein the milled grain does not comprise a heterologous cellulase.
